(19) United States
(12) Patent Application Publication
(10) Pub. No.: US 2016/0010127 A1
(43) Pub. Date: Jan. 14, 2016

(54) METHOD FOR ENZYMATIC HYDROLYSIS OF CELLULOSE

(71) Applicant: Agency for Science, Technology and Research, Singapore (SG)

(72) Inventors: Jinchuan WU, Singapore (SG); Zhibin LIU, Singapore (SG); Han Ling CHOW, Singapore (SG)

(21) Appl. No.: 14/379,075

(22) PCT Filed: Feb. 18, 2013
(86) PCT No.: PCT/SG2013/000064

§ 371 Date: Aug. 15, 2014

(30) Foreign Application Priority Data
Feb. 17, 2012 (SG) 201201135-9
May 31, 2012 (SG) 201204018-4

(51) Int. Cl. C12P 19/14; C12P 7/56; C12P 19/02

(52) U.S. Cl. CPC C12P 19/14; C12P 19/02; C12P 7/56; C12P 2203/00

(57) ABSTRACT

The present invention relates to methods for enzymatic hydrolysis of cellulose using whole cell cultures. The cellulase-producing microbes (e.g. fungus) may be cultivated at a lower temperature (e.g. about 30° C.) to produce extracellular cellulase enzymes followed by raising the temperature to a higher level (e.g. about 50° C.) to deactivate the cells and to promote the cellulose hydrolysis by extracellular cellulases resulting in continuous hydrolysis of cellulose to glucose without the risk of glucose being consumed by the deactivated cells.

The present invention relates to a method for enzymatic hydrolysis of cellulose using whole cell cultures.

### BACKGROUND OF THE INVENTION

Lignocellulose, composed of cellulose (30-50%), hemicellulose (20-40%) and lignin (10-30%), is the most abundant renewable resource on earth. Large-scale production of fuels and chemicals from this low-cost sustainable feedstock would provide significant environmental, economical and social benefits. However, lignocellulose cannot be directly utilized by most microorganisms of commercial interest as the cellulose and hemicellulose are tightly bound together by lignin to prevent them from being attacked, by microbes and needs to be pretreated to obtain fermentable sugars.

During pretreatment, hemicellulose is usually effectively degraded to fermentable sugars (D-xylose, L-arabinose and D-glucose) due to its lower polymerization degree but cellulose is hardly degraded and needs to be further subjected to acid or enzymatic hydrolysis to convert it into glucose before it can be utilized by microbes. Compared to acid-catalyzed hydrolysis of cellulose, enzymatic hydrolysis is more promising as it eliminates the use of large amount of chemicals, achieves high glucose yield and avoids the formation of inhibitory by-products.

Cellulose hydrolysis by enzymes is one of the bottlenecks limiting commercialization of lignocellulose-based biorefinery. Isolated cellulase enzymes are conventionally used for cellulose hydrolysis with at least 3 cellulase enzymes being needed for complete hydrolysis of cellulose. Cellulase, the enzyme that catalyzes cellulose hydrolysis to glucose, is actually a complex mixture of at least 3 key enzymes, endoglucanase (EC 3.2.1.4), exoglucanase (EC 3.2.1.91) and β-glucosidase (EC 3.2.1.21). Endoglucanase randomly attacks the internal bonds in cellulose chain and acts mainly on the amorphous cellulose. Exoglucanase hydrolyzes from the chain ends and produces predominantly cellobiose, which is further cleaved to two glucose molecules by β-glucosidase.

A key factor that hinders the commercialization of enzymatic cellulose hydrolysis is the high cost of cellulase enzymes. Attempts have been made to reduce the cost of enzymatic hydrolysis by either producing cheap cellulase enzymes by microbial cultivation or by recycling and reusing the enzymes after hydrolysis.

Enzymatic hydrolysis of cellulose has been extensively studied to develop cost-effective processes over the past decades. A lot of effort has been made to lower the cost of cellulases by improving the activities of individual cellulases, optimizing their ratios, recycling and reusing the enzymes. Novozymes and Genencor have claimed to reduce the cellulase cost by a factor of 10 from the original enzyme cost of US$4-5/gallon. Even so, the cost of cellulase enzyme is still too high to be accepted for industrial applications. Less attention has been paid to utilizing whole cell cultures for enzymatic hydrolysis of cellulose due to the difficulty in getting enough glucose using whole cells which will not produce more glucose than they need under the normal conditions.

There is a need to provide a method for enzymatic hydrolysis of cellulose that overcomes, or at least ameliorates, one or more of the disadvantages described above.

### SUMMARY

A first aspect of the invention provides a method for enzymatic hydrolysis of cellulose using whole cell cultures of cellulase-producing microbes, wherein the method comprises the following steps:
a) cultivating the cellulase-producing microbes at a first temperature to produce cellulases;
b) deactivating the celluase-producing microbes at a second temperature, the second temperature being at a higher temperature level than the first temperature; and
c) hydrolysing cellulose to produce glucose using said cellulases.

Advantageously, the disclosed method in one embodiment may provide a 1-pot, 2-step process for enzymatic hydrolysis of cellulose using whole cell cultures. The cellulase-producing microbes (e.g. fungus) are cultivated at a lower temperature (e.g. about 30° C.) to produce extracellular cellulase enzymes followed by raising the temperature to a higher level (e.g. about 50° C.) to deactivate the cells and to promote the cellulose hydrolysis by extracellular cellulases resulting in continuous hydrolysis of cellulose to glucose without the risk of glucose being consumed by the deactivated cells.

After (or simultaneously with) the hydrolysis, the whole broth contents including the released glucose and dead microbial (e.g. fungal) cells are able to be utilized as carbon and nitrogen sources for microbial fermentation to produce value-added chemicals. The whole process is able to be conducted in one pot without the requirement of any separation steps.

In one embodiment, Trichoderma reesei RUT-C30 may be used as the cellulase producer in the disclosed method. In this embodiment, 51% of the total cellulose (60 g/L) may be converted to glucose giving a final glucose concentration of 31.2 g/L, for example. In another embodiment, the process of cellulase production followed by simultaneous cellulose hydrolysis and fermentation using a thermophilic lactic acid bacterium Bacillus coagulans JI12 may convert 71% of the total cellulose (100 g/L) to lactic acid, giving a final lactic acid concentration of 79.2 g/L.

Advantages which may be achieved by the disclosed method include one or more (and preferably) all of the following:
1. Whole cell cultures may be utilized to catalyze the hydrolysis of cellulose without the requirement of isolating the cellulase enzymes from the broth;
2. The enzyme production and cellulose hydrolysis may be conducted in 1 pot by 2-steps by simply changing the temperatures in the pot (and optionally adding cellulose to the reaction broth);
3. The nutrients added for cultivating the whole cells for cellulase production can also be utilized for the subsequent fermentations.

Another advantage which may be achieved with the disclosed method is that the process cost of enzymatic hydrolysis of cellulose is expected to be significantly reduced compared to existing technology.

### Definitions

This section is intended to provide guidance on the interpretation of the words and phrases set forth below (and where appropriate grammatical variants thereof). Further guidance on the interpretation of certain words and phrases as used herein (and where appropriate grammatical variants thereof) may additionally be found in other sections of this specification.

As used herein, the singular forms “a”, “an” and “the” include the plural references unless the content clearly dictates otherwise. Thus for example, a reference to a composition containing “a compound” includes a reference to a single compound, and to two or more compounds (including mixtures of two or more compounds). It should be noted that the term “or” is generally employed in the sense including “and/or” unless the context dictates otherwise.

As used herein, the term “about” as used in relation to a numerical value means, for example, within 50% (±50%) of the numerical value, preferably ±30%, ±20%, ±15%, ±10%, ±7%, ±5%, ±2.5% or ±1%. Where necessary, the word “about” may be omitted from the definition of the invention.

In the context of this specification, the term “comprising” means “including. Thus, for example, a composition or polypeptide “comprising” X may consist exclusively of X or may include one or more additional components. In some embodiments, “comprising” means “including principally, but not necessarily solely”.

The terms “mutant” and “mutation” include any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence.

The terms “polypeptide” and “protein” are used interchangeably and include any polymer of amino acids (dipeptide or greater) linked through peptide bonds or modified peptide bonds, whether produced naturally or synthetically.

The word “substantially” does not exclude “completely” e.g. a composition which is “substantially free” from Y may be completely free from Y. Where necessary, the word “substantially” may be omitted from the definition of the invention.

### DETAILED DESCRIPTION OF THE INVENTION

The present invention may provide a 1-pot, 2-step process for enzymatic hydrolysis of cellulose using whole cell cultures. More specifically, one or more cellulase-producing micro-organisms (e.g. a fungus) may be cultivated at a lower temperature (e.g. about 30° C.) using cellulose as the substrate (or alternatively or additionally hemicellulose, holocellulose or lignocelluloses) to produce extracellular cellulase enzymes. Once the activity of the cellulases reaches a higher level, the broth temperature is raised to a higher level (e.g. about 50° C.) at which temperature the micro-organisms (e.g. fungal cells) stop growing or exhibit inhibited growth while the extracellular cellulases show their optimal activity, resulting in continuous hydrolysis of cellulose to glucose without the risk of glucose being consumed by the deactivated microbes .(e.g. fungal cells) for their growth. After (or simultaneously with). the hydrolysis, the whole broth contents including the released glucose and dead microbial (e.g. fungal cells) are able to be utilized as carbon and nitrogen sources for microbial fermentation to produce value-added chemicals. The whole process is able to be conducted in one pot without the requirement of any separation steps.

A first aspect of the invention provides a method for enzymatic hydrolysis of cellulose using whole cell cultures of cellulase-producing microbes, wherein the method comprises the following steps:
a) cultivating the cellulase-producing microbes at a first temperature to produce cellulases;
b) deactivating the. celluase-producing microbes at a second temperature, the second temperature being at a higher temperature level than the first temperature; and
c) hydrolysing cellulose to produce glucose using said cellulases.

In at least some embodiments, step c) comprises adding more cellulose into the broth for hydrolysis by said cellulases. However, if the cellulose added at the beginning of the process is sufficiently high to leave enough cellulose for the hydrolysis in step c) then additional cellulose need not be added in step c). However, only adding to the culture broth a suitable amount of cellulose in the beginning for producing cellulases then supplementing more cellulose in step c) is preferred as it is expected to reduce the substrate inhibition for producing cellulase enzymes. As will be appreciated from the discussion below the cellulose which is added to the culture broth (whether at the beginning of the process or in step c)) may be provided in various forms, for example in the form of cellulose, hemicellulose, holocellulose or lignocellulose. See also the discussion below regarding cellulose-containing materials: these materials may be used to provide the cellulose at the beginning of the process (i.e. for the cellulase production step) and/or the cellulose which is added to the culture broth in step c).

In at least some embodiments, step c) can be performed at the same higher temperature level of step b). The higher temperature level is such that the microorganisms stop growing or exhibit inhibited growth while the extracellular cellulases show their optimal activity. It is preferred that steps b) and c) are performed at the same temperature for simplicity and efficiency. However, in other embodiments steps b) and c) may be performed at different temperatures. For example, where step c) comprises the production of ethanol using Saccharomyces (e.g. S. cerevisiae) it is preferred that step b) is conducted at about 50° C. while step c) is conducted at about 30° C., which is the optimal temperature for Saccharomyces cerevisiae.

It will be appreciated from the foregoing that in at least some embodiments of the first aspect of the invention, there is provided a method for enzymatic hydrolysis of cellulose using whole cell cultures of cellulase-producing microbes, wherein the method comprises the following steps:
a) cultivating the cellulase-producing microbes at a first temperature to produce cellulases;
b) deactivating the celluase-producing microbes at a second temperature, the second temperature being at a higher temperature level than the first temperature; and
c) hydrolysing cellulose to produce glucose using said cellulases.

### The Substrate

The method of the first aspect of the invention relates to the enzymatic hydrolysis of cellulose. The cellulose which is hydrolysed may be provided in the form of one or more various substrates. For example, the cellulose which is hydrolysed may be provided in the form of one or more of the following substrates: cellulose, hemicellulose, holocellulose or lignocellulose. The one or more substrates which is/are used may be used in its unmodified state or pre-treated, such as with chemicals, or physical factors such as temperatures or enzymes. Examples of suitable cellulose-containing materials which may be used in the methods of the invention include one or more of the following: wood, wood residues, wood-related materials (e.g. saw dust) plant residues, corn stover, corn fiber, rice fiber, paper and paper products, wheat straw, oat hulls, brewers spent grains, pulp and paper mill waste, forestry waste, agricultural waste, bagasse, barley straw, cellulose, hemicellulose, holocellulose, lignocelluloses, empty fruit bunches of oil palm trees, municipal woody wastes, and mixtures of any of the foregoing.

### The Cellulase-Producing Microbes

Advantageously, the present invention is able to utilise whole cell cultures of cellulase-producing microbes. The whole cell cultures can be used directly to hydrolyse cellulose without separating hydrolytic enzymes such as cellulase (whether 1, 2 and/or all 3 of endoglucanase (EC 3.2.1.4), exoglucanase (EC 3.2.1.91) and β-glucosidase (EC 3.2.1.21)) from whole cells (such as the cellulase-producing microbial cells). Preferably, the whole cell cultures are used directly to hydrolyse cellulose without any separation steps.

In at least some embodiments, the microbes are cellulase producers, hemicellulase producers or ligninase producers. Cellulase-producing microbes sometimes also produce hemicellulases and/or ligninases and, accordingly, the broth (reaction medium) may comprise hemicellulase producers and/or ligninase producers. Cellulase-proudcing microbes can commonly produce all the lignocellulose-degrading enzymes including cellulases, hemicellulases and ligninases. Thus, in at least some embodiments of the invention the cellulase-producing microbes are able to degrade lignocellulose and produce cellulases, hemicellulases and ligninases.

The term “hemicellulase producers” includes a reference to microorganisms that are able to produce any hemicellulases that cleave hemicellulose to release oligosaccharides and sugars, including endoxylanase, exoxylanase, Beta-xylosidase, α-glucuronidase, α-arabinofuranosidase, arabinase, endo-mannanase, β-mannosidase, acetyl xylan esterase and feruloyl xylan esterase.

The term “ligninase producers” includes a reference to microorganisms that are able to produce the enzymes responsible for lignin degradation, including lignin peroxidase, Manganese peroxidase and laccase.

As mentioned above, cellulase, the enzyme that may in one embodiment catalyze cellulose hydrolysis to glucose, is actually a complex mixture of at least 3 key enzymes, endoglucanase (EC 3.2.1.4), exoglucanase (EC 3.2.1.91) and β-glucosidase (EC 3.2.1.21). Unless the context indicates otherwise, the term “cellulase” as used herein includes a reference to one, two or all three of: endoglucanase (EC 3.2.1.4), exoglucanase (EC 3.2.1.91) and β-glucosidase (EC 3.2.1.21). Thus, a cellulase-producing microbe includes a reference to a microbe which produces one, two or all three of: endoglucanase (EC 3.2.1.4), exoglucanase (EC 3.2.1.91) and β-glucosidase (EC 3.2.1.21).

The cellulase-producing microbes which are used in step a) of the methods of the present invention individually or collectively produce the whole spectrum of cellulases which, within the meaning of the present invention, means at least the following 3 enzymes: endoglucanase (EC 3.2.1.4), exoglucanase (EC 3.2.1.91) and β-glucosidase (EC 3.2.1.21). These 3 enzymes are produced extracellularly by the cellulase-producing microbes employed in the various aspects of the invention.

The cellulase-producing microbes may comprise one or more different strains of cellulase-producing microbe. Where the cellulase-producing microbes consist of a single strain of microbe, then the whole spectrum of cellulases is produced by that single strain of microbe. Alternatively, the whole spectrum of cellulases may be provided by two or more different strains of cellulase-producing microbes. Where the whole spectrum of cellulases is produced by two or more strains of cellulase-producing microbes, one or more (or all) of the two or more cellulase-producing microbes may individually produce the whole spectrum of cellulases with the other cellulase-producing microbe(s) producing at least one of endoglucanase, exoglucanase and β-glucosidase. In an alternative embodiment, none of the two or more different strains of cellulase-producing microbe individually produces the whole spectrum of cellulases, but collectively the two or more microbe strains produce the whole spectrum of cellulases (e.g. one microbe strain may produce two of the three cellulases, whilst a second microbe strain produces the third cellulase; or each of the three cellulases is produced by a different strain of microbe etc.).

In a preferred embodiment, the cellulase-producing microbes used in the methods of the present invention comprises a single strain of microbe which produces the whole spectrum of cellulases; optionally one or more further cellulase-producing microbes may be present. In one embodiment, the cellulase-producing microbes used in the present invention consist of a single strain of cellulase-producing microbe which produces the whole spectrum of cellulases.

In another embodiment, the cellulase-producing microbes used in the methods of the present invention is a mixture of several strains of cellulase-producing microbes. By “several” we include a reference to 2 or more, 3 or more, or 4 or more.

The one or more cellulase-producing microbes which are used in the methods of the present invention may be any organism which may be used to produce cellulase under the conditions of step a) of the methods of the present invention. Thus, the one or more cellulase-producing microbes which are used in the methods of the present invention may be used to produce cellulases at the first (lower) temperature used in step a). Whilst the one or more cellulase-producing microbes used in the methods of the present invention may be used to produce cellulases at the lower temperature of step a), the cellulase activity is generally greater at the higher temperature level that may be associated with step c) in the disclosed method, than at the lower temperature of step a). Suitably, the optimal cellulase activity is in a temperature range of from about 45-100° C., preferably from about 50-100° C., more preferably from about 50-80° C., from about 50-70° C., or from about 50-60° C. In a preferred embodiment, the cellulase activity is optimal from about 45-55° C., and preferably at about 50° C. Most cellulase-producing fungi survive and produce cellulases only under mesophilic conditions but the produced cellulases are most active under thermophilic conditions. Methods for evaluating cellulase activity are well known in the art (see e.g. the method used in the section entitled “Analytical Methods” in the Examples section).

In step b) of the methods of the present invention the one or more cellulase-producing microbes which are used in the methods of the present invention are deactivated by the higher temperature level. Accordingly, the one or more cellulase-producing microbes are not heat tolerant, that is they have stopped growing (e.g. because they are dead) or their growth is greatly or substantially inhibited, by the higher temperature level of step b) of the present invention.

In at least some embodiments of the invention, at least one or more (and optionally all) of the one or more cellulase-producing microbes are fungi, such as filamentous fungi or yeast.

Some bacteria are also able to produce cellulases, hemicellulases and ligninases and, accordingly, in at least some embodiments of the invention, at least one or more (and optionally all) of the one or more cellulase-producing microbes are bacteria.

In at least some embodiments of the invention, the bacteria which could be used to produce cullulases, particularly thermostable cellulases, include Clostardium species and Bacillus species.

The term filamentous fungi refers to all filamentous forms of the subdivision Eumycotina. Examples of filamentous fungi which may be used in the present invention may include, for example: Trichoderma sp., (e.g. Trichoderma reesei, such as Trichoderma reesei RUT-C30 (ATCC 56765) or a variant derived therefrom), Penicillium sp. (e.g. Penicillium funiculosum. P. janthinellum), Humicola sp. Chrysosporium sp., Gliocladium sp., Aspergillus sp. (e.g., A. oryzae, A. niger, A. nidulans, A. phoenicis and A. awamori), Fusarium sp.,Neurospora sp., Hypocrea sp., Mucor, and Emericella sp.

Preferably, at least one or more of (and optionally all of) the one or more cellulase-producing microbes are selected from the group consisting of: Trichoderma sp., (e.g. Trichoderma reesei, such as Trichoderma reesei RUT-C30 (ATCC 56765) or a variant derived therefrom), Aspergillus sp. (e.g., A. oryzae, A. niger, A. nidulans, and A. awamori), and Penicillium sp. (e.g. Penicillium funiculosum).

Preferably, at least one or more of (and optionally all of) the one or more cellulase-producing microbes are selected from the group consisting of: Trichoderma reesei (e.g. Trichoderma reesei RUT-C30 (ATCC 56765) or a variant derived therefrom), Aspergillus niger and Penicillium funiculosum, Clostridium species (e.g. Clostridium cellulolyticum) and Bacillus species (e.g. Bacillus subtilis).

Preferably, at least one or more of the one or more cellulase-producing microbes is Trichoderma reesei RUT-C30 (ATCC 56765) or a variant derived therefrom. Optionally, Trichoderma reesei RUT-C30 (ATCC 56765) (or a variant derived therefrom) is co-cultured in step a) with a cellulase-producing microbe which is a β-glucosidase producer. In this way cellulase activity may be increased as compared to using the single culture alone. By co-culturing Trichoderma reesei RUT-C30 (ATCC 56765) or a variant derived therefrom with a β-glucosidase producer the glucose yield produced in step a) may be improved. The β-glucosidase producer may optionally be an Aspergillus species, e.g. A. Phoenicis.

In one embodiment of the invention, the cellulase-producing microbes consist of Trichoderma reesei RUT-C30 (ATCC 56765) (or a variant derived therefrom) and one or more cellulase-producing microbes which produce β-glucosidase.

In one preferred embodiment, the cellulase-producing microbes are Trichoderma reesei RUT-C30 (ATCC 56765) or a variant derived therefrom.

The term “variant” as used herein in relation to Trichoderma reesei RUT-C30 (ATCC 56765) includes Trichoderma reesei which vary in one or more respects from Trichoderma reesei RUT-C30 (ATCC 56765). The term “variant” encompasses “mutants” but also encompasses microbes having one or more genetic modifications created by techniques/phenomena other than by mutagenesis, such as by recombinant technology. The variants are derived from Trichoderma reesei RUT-C30 (ATCC 56765) and the phrase “derived from”/“derived therefrom” is intended to be construed broadly. Thus, the term would include, for example, variants which have been derived directly from Trichoderma reesei RUT-C30 (ATCC 56765) (i.e. from Trichoderma reesei RUT-C30 (ATCC 56765) per se and not from, say, a variant or mutant of Trichoderma reesei RUT-C30 (ATCC 56765)) as well as variants which have been indirectly derived from Trichoderma reesei RUT-C30 (ATCC 56765) (e.g. variants which have been derived from a mutant or variant of Trichoderma reesei RUT-C30 (ATCC 56765), which mutant or variant has itself been derived from Trichoderma reesei RUT-C30 (ATCC 56765)).

In at least some embodiments of the invention, a variant derived from Trichoderma reesei RUT-C30 (ATCC 56765) is a mutant derived Trichoderma reesei RUT-C30 (ATCC 56765). The phrase “mutant derived from” is intended to be construed broadly. Thus, the term would include, for example, mutants which have been derived directly from Trichoderma reesei RUT-C30 (ATCC 56765) (i.e. from Trichoderma reesei RUT-C30 (ATCC 56765) per se and not from, say, a variant of Trichoderma reesei RUT-C30 (ATCC 56765)) as well as mutants which have been indirectly derived from Trichoderma reesei RUT-C30 (ATCC 56765) (e.g. mutants which have been derived from a mutant or variant of Trichoderma reesei RUT-C30 (ATCC 56765), which mutant or variant has itself been derived from Trichoderma reesei RUT-C30 (ATCC 56765)).

Techniques for creating variants and mutants will be well known to those skilled in the art, e.g. exerting selective pressure for improved or new characteristics, using gene replacement techniques or classical chemical mutagenesis. In some embodiments, the genome of Trichoderma reesei RUT-C30 (ATCC 56765) (or a variant or mutant thereof) may be augmented with genetic material (e.g. with one or more “control sequences” (e.g. promoters, terminators etc.) and/or with one or more protein-encoding sequences) to create a variant. In this way, variants may be created which express one or more (e.g. at least 1, 2, 3, 4 or 5) heterologous proteins.

The variants derived from Trichoderma reesei RUT-C30 (ATCC 56765) retain the ability of Trichoderma reesei RUT-C30 (ATCC 56765) to produce the whole spectrum of cellulases under the culture conditions used in step a) of the methods of the present invention and the endoglucanase, cellobiohydrolase and β-glucosidase so-produced may then be used in step c) of the methods of the present invention to achieve cellulose hydrolysis. Like Trichoderma reesei RUT-C30 (ATCC 56765), the variants derived from Trichoderma reesei RUT-C30 (ATCC 56765) are also deactivated by the higher temperature level used in step b) of the methods of the present invention.

As with Trichoderma reesei RUT-C30 (ATCC 56765), the variants may optionally be co-cultured with a β-glucosidase producer.

In at least some embodiments of the invention at least one or more of (and optionally all of) the one or more cellulase-producing microbes are yeast cells. Yeasts do not form an exact taxonomic or phylogenetic grouping but rather it is the colloquial name for single-celled members of the fungal divisions Ascomycota and Basidiomycota. The budding yeasts (“true yeasts”) are classified in the order Saccharomycetales. Most reproduce asexually by budding, although a few do so by binary fission. Yeasts are unicellular, although some species with yeast forms may become multicellular through the formation of a string of connected budding cells known as pseudohyphae, or false hyphae as seen in most molds. Examples of cellulase-producing yeasts include those which have been engigneered by Prof. Akihiko Kondo's group in Kobe University and which produce cellulases and hemicellulases on the cell surface.

The cellulase-producing microbes used in the present invention may optionally be isolated from nature or obtained. from a culture collection.

### Culturing the Cellulase-Producing Microbes to Produce Cellulases

In step a) of the methods of the present invention the cellulase-producing microbes are cultivated at a lower temperature to produce cellulases.

As used herein, the terms “culture” and “cultivate” (and for the avoidance of doubt, grammatical variants thereof) are used interchangeably.

The skilled person will readily be able to arrive at suitable culture conditions (see e.g. Ghose T. K, Sahai V. Production of cellulases by Trichoderma reesei QM 9414 in fed-batch and continuous-flow culture with cell recycle. Biotechnol. Bioeng., 21: 283-296 (1979); and Singhania R. R, Sukumaran R. K, Pandey A. Improved cellulase production by Trichoderma reesei RUT C30 under SSF through process optimization, Appl. Biochem. Biotechnol., 142: 60-70 (2007)). Suitable culture conditions may include liquid or solid media and may, for example, include shake flask cultivation, small scale or large scale fermentations (including continuous, batch and fed batch fermentations) in laboratory or industrial fermentors, with suitable medium containing physiological salts and nutrients. Suitable media are available from commercial suppliers or may be prepared according to published compositions (e.g., in catalogues of the American Type Culture Collection). Examples of suitable culture media may include modified YPD medium with no content or minimized content of glulose and suitable amout of cellulose substances with or without addition of surfactants such as Tween 80. Growth factors, amino acids or other additives (e.g. antibiotics for selective pressure) may be added to such culture media as desired or necessary. Other considerations, may comprise temperature and pH of the growth medium.

The cellulase-producing microbes are cultivated at a first temperature, or a “lower temperature”, to produce cellulases. The first temperature, or a “lower temperature”, is selected from a temperature range of from about 10-50° C., more preferably from about 15-45° C., still, more preferably from about 20-40° C., yet more preferably from about 25-35° C., and even more preferably from about 27-33° C. In a preferred embodiment, the temperature is about 30° C. The terms “lower temperature” and “first temperature” as used herein may be used interchangeably.

Suitably, the one or more cellulase-producing microbes are mesoophilic micro-organisms. By a “mesoophilic micro-organism”, it includes a reference to an organism which thrives at said “lower temperature level”. Thus, a mesophilic organism refers to an organism which thrives at a temperature of about 10-50° C., more preferably from about 15-45° C., still more preferably from about 20-40° C., yet more preferably from about 25-35° C., and even more preferably from about 27-33° C.

### Raising the Temperature of the Reaction Broth

In step b) of the methods of the present invention the broth temperature is raised to a second or a “higher temperature level” to thereby deactivate the cellulase-producing microbes.

Step b) may be performed after the cellulase activity (the synergistic activity of the 3 cellulase enzymes) reaches a higher level, i.e. after the cellulase-producing microbes have been cultivated for a suitable time period at the lower temperature to allow for the production of the cellulases. In at least some embodiments of the invention, the term “a higher level” as used herein in relation to cellulase activity includes a reference to where there is 60-100% (preferably, at least 65%, 70%, 75%, 80%, 90% or 95%) of the maximal cellulase activity. The optimal cellulase activity is measured under the optimal pH and temperature.

Those skilled in the art will be able to determine a suitable time period for the cultivation of the cellulase-producing microbes to produce the cellulases but it will be appreciated that it will be influenced by a number of factors, including the microbe or microbes used, and the amount of cellulose carbon source used for producing cellulases etc. In at least some embodiments, the cellulase-producing microbes are cultivated at least until the increase in cellulase activity starts to slow down. Methods for assaying cellulase activity are known in the art (and see e.g. the method used in the section entitled “Analytical Methods” in the Examples section). Cellulase activity (the synergistic activity of the 3 cellulase enzymes) may optionally be measured by detecting the total amount of reducing sugars using the DNS method.

Preferably, the cellulase-producing microbes are cultivated in accordance with step a) for between 24 hours to 15 days. Preferably, the cellulase-producing microbes are cultivated in accordance with step a) for at least 24 hours, 48 hours, 72 hours, 96 hours, 120 hours, 144 hours, 168 hours, 192 hours, 216 hours, 240 hours, 264 hours, 288 hours, 312 hours, 336 hours, 360 hours or 384 hours.

By a “higher temperature level” or “a second temperature”, it includes a temperature range of from about 45-100° C., preferably from about 50-100° C., more preferably from about 50-80° C., yet more preferably from about 50-70° C., and even more preferably from about 50-60° C. In a preferred embodiment, the temperature is from about 45-55° C., and preferably at about 50° C.

Steps b) and c) of the present invention may be both performed at a higher temperature level as compared to step a). As discussed above, the higher temperature level used in steps b) and c) may be the same or different. Thus, there may be a first higher temperature used in step b) and a second higher temperature used in step c). The first higher temperature and second higher temperature may be independently selected from about 45-100° C., preferably from about 50-100° C., more preferably from about 50-80° C., yet more preferably from about 50-70° C., and even more preferably from about 50-60° C. In a preferred embodiment, the higher temperature level (or the first higher temperature and second higher temperature) may be selected from about 45-55° C. In yet a preferred embodiment, the higher temperature level is preferably about 50° C.

The higher temperature level deactivates the cellulase-producing microbes. The term “deactivated” (and for the avoidance of doubt, grammatical variants thereof) as used herein in relation to microbial cells includes a reference to microbial cells which have stopped growing (the cells may be dead or alive) or whose growth has been greatly or substantially inhibited. The microbial cells may auto-lyze to thereby release nutrition for the fermentative micro-organisms in the fermentation step. By deactivating microbial cells their consumption of the products of the hydrolysis of cellulose (e.g. glucose) may be stopped.

### Cellulose Hydrolysis

In step c) of the methods of the present invention cellulose is optionally added into the broth which is then hydrolysed by the cellulases to produce glucose. The addition of cellulose in step c) may occur at a single time point or at one or more time points. Cellulose may be added at the same time as the temperature is raised in step b) and/or after the temperature has been raised (i.e. after step b)). Alternatively or additionally, cellulose may be added prior to (e.g. immediately prior to) step b) although, it will be appreciated that it will be generally disadvantageous for cellulose to be added before the cellulase-producing microbes have been deactivated since the cellulase-producing microbes may consume the cellulose.

The cellulose hydrolysis step of step c) is performed at a a higher temperature level. As discussed above, the higher temperature level used in steps b) and c) may be the same or different.

### Preferred Features of the Method

Steps a), b) and c) of the method of the first aspect of the invention may be performed without separating cellulase enzymes from the cellulase-producing cells. Preferably, steps a), b) and c) of the method of the first aspect of the invention are performed without any separation of enzymes from cells. Preferably, steps a), b) and c) of the method of the first aspect of the invention are performed without any separation steps. Steps a), b) and c) can be performed in “1-pot”, i.e. in a single reactor. The term “reactor” as used herein includes a reference to any vessel suitable for practicing a method of the present invention. The term “reactor” as used herein may be used interchangeably with the term “fermenter” and “reaction vessel”.

The method of the first aspect of the invention may be viewed as a two-step process with the first step being cultivating the cellulase-producing microbes at a lower temperature to produce cellulases. The second step is raising the temperature to a higher temperature level after the cellulase activity reaches a higher level to thereby deactivate the cells and to hydrolyse the cellulose to produce glucose, for example, at said higher temperature level. Thus, the first step is conducted at the lower temperature (production of cellulases) whilst the second step is conducted at the higher temperature (deactivation of the cellulase-producing microbes and hydrolysis of cellulose). Thus, the two steps may be performed by simply changing the temperature of the reaction broth. A schematic of the two-step process of the invention is shown in FIG. 4, with a third optional step also being shown which is the fermentation of glucose (to lactic acid in the example shown in FIG. 4). The third step may also be conducted in the same reaction vessel. Moreover, as discussed below the third optional step may be performed simultaneously with the second step of the invention (SSF).

Because the present invention utilises whole cell cultures of cellulase-producing microbes directly to hydrolyse cellulose without any separation steps, it is not necessary to add enzymes such as cellulase enzymes (e.g. in the form of isolated or purified enzymes, cells producing enzymes, whole broth from enzyme production etc.), to the reaction broth. The term “isolated” as used herein, includes a reference to material that is substantially or essentially free from components that normally accompany it in its native state. The term “isolated” does not denote the method by which an isolated enzyme is obtained or the level of purity of the preparation. Thus, isolated enzymes may be produced recombinantly, or isolated directly from cells etc.

Thus, in at least some embodiments of the invention endoglucanase, cellobiohydrolase and/or β-glucosidase is/are not added to the reaction broth (e.g. in the form of isolated or purified enzymes, cells producing enzymes, whole broth from enzyme production etc.). Thus, the only cellulases present in the broth will be those produced by the one or more cellulase-producing microbes, and, where the methods of the invention comprise a fermentation step, optionally also by the one or more fermenting micro-organisms (fermenting micro-organisms may occasionally produce a cellulase). It is also possible that some further cellulases may be present as “impurities”. For instance, it is possible that some thermotolerant cellulase-producing microbes may inadvertently be brought into the system together with the cellulose substrates or with other nutrients.

In at least some embodiments of the invention, endoglucanase, cellobiohydrolase and/or β-glucosidase is/are not added to the reaction broth (e.g. in the form of isolated or purified enzymes, cells producing enzymes, whole broth from enzyme production etc.): (i) before or during step a); (ii) after step a); (iii) during step b); (iv) after step b); (v) during step c); and/or (vi) after step c).

In at least some embodiments of the invention, endoglucanase, cellobiohydrolase and/or β-glucosidase is/are not added to the reaction broth (e.g. in the form of isolated or purified enzymes, cells producing enzymes, whole broth from enzyme production etc.) at the lower temperature of step a) and/or at the higher temperature level(s) of steps b) and c).

In at least some embodiments, the methods of the present invention do not comprise the addition of an enzyme to the reaction broth. Thus, the only enzymes present in the broth will be those produced by the one or more cellulase-producing microbes and, where the methods of the invention comprise a fermentation step, those enzymes which may be produced by the one or more fermenting micro-organisms. It is also possible that some further enzymes may be present as “impurities”. For instance, it is possible that some enzyme producing microbes (e.g. thermotolerant cellulase-producing microbes) may inadvertently be brought into the system together with the cellulose substrates or with other nutrients. However, it is envisaged that in many embodiments the added materials will be heat treated e.g. autoclaved before their addition and in these embodiments such enzyme-producing microbes would not be present in the broth.

### Optional Fermentation Step

In at least some embodiments, the method of the first aspect of the invention comprises a further step, step d). In step d) the glucose produced by the hydrolysis of the cellulose in step c) is fermented by one or more fermenting micro-organisms. The whole broth contents including the released glucose and the deactivated cellulase-producing microbes may be utilized as carbon and nitrogen sources for microbial fermentation to produce value-added chemicals. The nutrients added for cultivating the whole cells for cellulase production can also be utilized for the fermentation. Optionally, nutrients such as nitrogen sources, inorganic salts or trace elements may be added to the reaction broth for the fermentation step. Optionally, the air supply may be stopped and/or the pH may be adjusted.

Steps a), b) and c) and d) can be performed in “1-pot”, i.e. in a single reactor.

Preferably, steps a), b) c) and d) of the method of the first aspect of the invention are performed without any isolation of enzymes so that no isolated enzymes are added to the reaction broth. Preferably, steps a), b), c) and d) of the method of the first aspect of the invention are performed without any separation steps.

In at least some embodiments, the one or more fermenting micro-organisms is/are homofermentative micro-organisms. Homofermentative microorganisms include, for example, lactic acid producers which produce primarily lactic acid as the single product. The homofermentative lactic acid bacteria include Bacillus coagulans, Lactobacillus casei etc.

The fermentation product may include, for example: alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); enzymes; and vitamins (e.g., riboflavin, B12, beta-carotene).

In at least some embodiments, the fermentation produces lactic acid and the one or more fermenting micro-organisms comprises a lactic acid producing microorganism (preferably a lactic acid producing bacterium).

In at least some embodiments, the fermentation produces an alcohol (e.g. ethanol) and the one or more fermenting micro-organisms comprises an alcohol (e.g. ethanol) producing microorganism. Methods of alcohol fermentations are described in the Alcohol textbook, a reference for the beverage, fuel and industrial alcohol industries, 3rd ed., eds K. A. Jacques et al., 1999, Nottingham University Press, UK.

Step d) may be performed simultaneously with step c) (SSF) and/or after step c). Where step d) is performed simultaneously with step c) and after step c), step d) is said to overlap with step c) so that a first part of step d) is performed simultaneously with step c) and a second, subsequent part of step d) is performed after step c). Accordingly, it will be appreciated that embodiments are envisaged where step d) is performed at least partly (and optionally entirely) at the same time as step c).

Preferably, the one or more fermenting micro-organisms are thermophilic micro-organisms. By a “thermophilic micro-organism”, it includes a reference to an organism which thrives at said “higher temperature level”. Thus, a thermophilic organism refers to an organism which thrives at a temperature of about 45-100° C., preferably from about 50-100° C., more preferably from about 50-80° C., yet more preferably from about 50-70° C., even more preferably from about 50-60° C., from about 45-55° C., or at about. 50° C. The use of thermophilic micro-organisms is very suitable for applications in simultaneous saccharification and fermentation where the optimal temperatures of cellulose hydrolysis and fermentation can well match with each other, such as the lactic acid production from cellulose or hemicelluloses using thermophilic lactic acid bacteria. Thus, in a preferred embodiment at least one of the one or more fermenting micro-organisms is a thermophilic lactic acid bacterium, such as a Bacillus sp. (e.g. B. coagulans).

In a preferred embodiment, cellulose hydrolysis and glucose fermentation is conducted simultaneously by adding thermophilic lactic acid bacteria and supplementing nutrients to produce lactic acid.

### Product Recovery

Following fermentation, the fermentation product (e.g. lactic acid or alcohol) may optionally be recovered. Where alcohol is the fermentation product, then it may be recovered by, for example, distillation. Optionally, it may then be concentrated and/or further processed to render it more suitable/ready for its intended use. In one embodiment, the alcohol is ethanol. The ethanol may be used as, e.g., fuel ethanol, drinking ethanol, or as industrial ethanol.

Co-products of the fermentation process may also be obtained. Examples of co-products may include distillers' grains (e.g. distillers dried grain (DDG) and Distillers Dried Grains with Solubles (DDGS)), germ, gluten meal, fiber, corn oil, and high protein animal feed. Moreover the biomass remaining after fermentation could be used to produce biodiesel, and/or synthetic biofuels from products of thermochemical conversions and/or to generate heat. Thus, the methods of the invention may comprise recovering a coproduct from the fermentation process. Thus, the present invention further relates to generating biodiesel, biofuel, heat and/or carbon dioxide from the biomass remaining after fermentation.

Accordingly, a further step of the invention comprises step e) which is recovering one or more products (e.g. one or more fermentation products, one or more co-products, or one or more enzymes (see section below)) from the reaction broth.

A further aspect of the invention provides a product (e.g. a fermentation product (such as lactic acid or alcohol) or a co-product) obtained by a method of the invention.

### Recovery of Cellulase Enzymes

The methods of the present invention may further comprise after steps a) to c) (or when the method comprises a fermentation step then after steps a) to d)) the step of recovering one, two or three of the cellulases (i.e. one, two or all three of endoglucanase, cellobiohydrolase and β-glucosidase) from the reaction broth. In one embodiment, endoglucanase and/or cellobiohydrolase is/are recovered. In another embodiment, endoglucanase and/or β-glucosidase is/are recovered. In another embodiment, cellobiohydrolase and/or β-glucosidase is/are recovered.

The one, two or three cellulase enzymes may be recovered from the reaction broth using techniques known in the art (e.g. separating the cells from the medium by centrifugation or filtration (e.g. ultrafiltration), precipitating proteinaceous components of the medium by means of a salt such as ammonium sulphate, followed by the use of chromatography such as ion exchange, affinity chromatography, or the like). In a preferred embodiment, adsorption/desorption using cation exchange resins at different pHs is used. The cellulase from Trichoderma reesei has been successfully recovered by simple adsorption/desorption using cation exchange resins at different pHs achieving almost 100% of activity recovery with the supplement of β-glucosidase, which was other unable to be recovered due to its strong adsorption and poor desorption.

### BRIEF DESCRIPTION OF DRAWINGS

FIG. 1 Time courses of cellulase production at (30±1)° C. (A) and cellulose hydrolysis at (50±1)° C. (B) in shaking flasks. For (B), the strains. ATCC 56765 and ATCC 60675 were cultivated at 30° C. for 192 h and 288 h, respectively, followed by increasing the temperature to (50±1)° C. and adding 2 g of cellulose.

FIG. 2 Time courses of cellulase production, at (30±1)° C. (A) and cellulose hydrolysis at (50±1)° C. (B) in a 2 L fermenter containing 1 L culture medium. For cellulose hydrolysis, the strain ATCC 56765 was cultivated at (30±1)° C. for 67 h followed by increasing the, temperature to (50±1)° C. and adding 20 g of cellulose in the beginning and another 20 g of cellulose at 115 h.

FIG. 3 Time courses of simultaneous cellulose hydrolysis and fermentation to lactic acid at (50±1)° C. The strain ATCC 56765 was cultivated in a 2 L fermenter containing 1 L of culture medium at (30±1)° C. for 67 h followed by raising the temperature to (50±1)° C., adding 80 g of cellulose and essential nutrients, and inoculating the thermophilic lactic acid bacteria. The pH was controlled at 5.5 during the fermentation.

FIG. 4. Schematic of a 1 pot, 2-step process according to the present invention for cellulose hydrolysis.

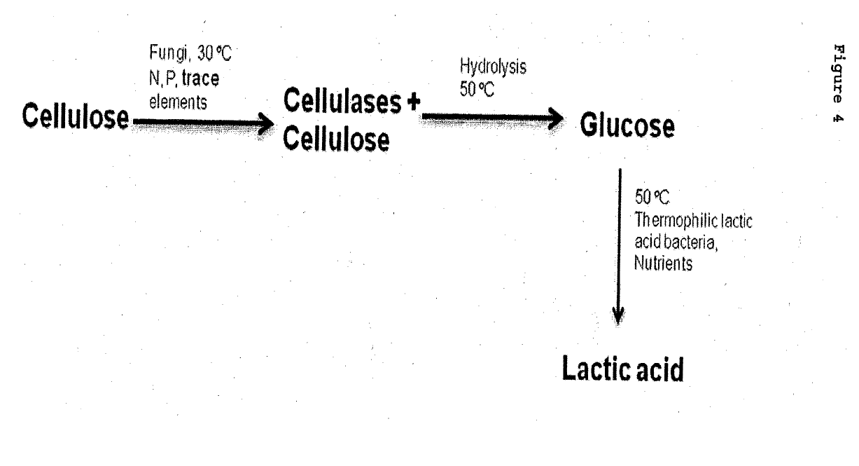

### EXAMPLES

### Example 1

### Material and Methods

### Microbes and Culture Media

Two cellulase-producing fungi, Trichoderma reesei RUT-C30 (ATCC 56765) and Drechslera dictyoides (ATCC 60575), were utilized. They were maintained on PDA plates at 4° C. and biweekly sub-cultured. Bacillus coagulans JI-n-12-1-1, a thermophilic lactic acid bacterium (optimal temperature 50° C.), was isolated from the Singapore environment by our lab and used to produce lactic acid. It is a homofermentative strain on both glucose and xylose and produces only L-lactic acid (ee>99%). The strain was maintained on MRS agar plates at 4° C.

Unless otherwise specified, the medium for cultivating the fungi for cellulase production contained (per liter) : 20 g of cellulose, 2 g of (NH4)₂SO₄, 10 ml of corn steep liquor, 1 g of K₂HPO₄, 2 g of KH₂PO₄, 0.2 g of MgSO₄·7H₂O, 0.5 g of NaCl and 1 ml of trace element solution. The trace element solution contained (per liter): 0.1 g of ZnSO₄·7H₂O, 0.01 g of H₃BO₃, 00.01 g of Na₂MoO₄·2H₂O, 0.1 g of CoCl₂·6H₂O, 0.1 g of CuSO₄·5H₂O, 0.1 g of FeSO₄·7H₂O, 0.5 g of MnSO₄·4H₂O and 0.1 g of CaCl₂·2H₂O. The medium was adjusted to the desired pH prior to autoclaving.

### Cellulase Production and Cellulose Hydrolysis in Shaking Flasks

To test the feasibility of the 1-pot, 2-step process for enzymatic hydrolysis of cellulose using whole cell cultures, cellulase production and cellulose hydrolysis were tested respectively in 250 ml flasks containing 100 ml of culture medium at pH7.5.

For cellulase production, the inoculum (2 ml) was prepared by inoculating a spoon of the fungal cells from PDA plates into 5 ml of culture medium of pH 7.5 and incubating at (30±1)° C. for 16 h with shaking at 250 rpm. After being inoculated, the flasks were incubating at (30±1)° C. and 250 rpm. Liquid samples (1.5 ml) were regularly removed from the flasks and centrifuged at 12000 rpm for 10 min. The supernatant was collected for assay of cellulase activity.

For cellulose hydrolysis, the inoculum was prepared, inoculated into the flasks and cultured at (30±1)° C. as described above until when the cellulase activity reached the highest. Afterwards, 2 g of cellulose was added and the flasks were incubated at (50±1)° C. with shaking at 250 rpm. Liquid samples (1.5 ml) were regularly removed from the flasks and centrifuged at 12000 rpm for 10 min. The supernatant was collected for analysis of sugars by HPLC.

### Cellulase Production and Cellulose Hydrolysis in Fermenters

The cellulase production and cellulose hydrolysis were also respectively conducted in a 2 L fermenter containing 1 L culture medium of pH 5.0. For cellulase production, the seed fungi were prepared in 100 ml culture medium at (30±1)° C. for 3 days and 10% (v/v) of inoculum was applied. During fermentation, the temperature was maintained at (30±1)° C., the pH was controlled at 3.75 for 48 h and 3.5 afterwards and the stirring rate was kept at 300 rpm. Filter-sterilized air was continuously bubbled at 0.3 L/min to maintain a PO₂ of 20%. Liquid samples (5 ml) were taken at predetermined time intervals and centrifuged at 4000 rpm for 10 min. The supernatants were collected and properly diluted for cellulase activity assay.

For cellulose hydrolysis, the inoculum was prepared, inoculated into the fermenter and cultured at (30±1)° C. as described above until when the cellulase activity reached the highest. Afterwards, 20-80 g/L of cellulose was added and the temperature was raised to (50±1)° C. Liquid samples (5 ml) were taken at predetermined time intervals and centrifuged at 4000 rpm for 10 min. The supernatants were collected and properly diluted for sugar analysis by HPLC.

### Cellulase Production, Simultaneous Cellulose Hydrolysis and Fermentation to Lactic Acid in a Fermenter

The experimental procedures for cellulase production in a 2 L fermenter containing 1 L culture medium at pH5.0 were the same as described above until when the cellulase activity reached the highest. Afterwards, 80-100 g/L of cellulose was added, the temperature was raised to 50° C., the air supply was stopped and the pH was adjusted to 5.5. Then 20 g/L of yeast extract, 2 g/L of (NH₄)₂SO₄, 2 g/L of KH₂PO₄, 0.05 g/L MnSO₄ and 0.01 g/L FeSO₄ were added, followed by inoculation of 10 ml of sterile distilled water containing the seed lactic acid bacterium Bacillus coagulans JI-n-12-1-1, which was prepared by cultivating Bacillus coagulans JI-n-12-1-1 in 100 ml of LB medium at (50±1)° C. for 24 h, removing the supernatant by centrifugation at 4000 rpm for 20 min and dispersing the cells in 10 ml of sterile distilled water. Liquid samples (5 ml) were taken at predetermined time intervals and centrifuged at 4000 rpm for 10 min. The supernatants were collected and properly diluted for sugar and lactic acid analysis by HPLC.

### Analytical Methods

The cellulase activity was assayed by mixing 0.1 ml of culture supernatant with 1 ml of 1% carboxymethyl cellulose (CMC) in 0.1 M sodium acetate buffer of pH 4.8 at 50° C. for 30 min. The released reducing sugar was analyzed. by the 3, 5-dinitrosalicylic acid (DNS) method.

The concentrations of glucose and lactic acid were analyzed by HPLC (Shimadzu, model LC10ATvp) with a Bio-rad Aminex HPX-87H (300 mm×78 mm) column at 50° C. Samples (20 μl) were eluted by 5 mM H₂SO₄ at 0.6 ml/min and detected by a refractive index detector.

### Results

### Cellulase Production and Cellulose Hydrolysis in Shaking Flasks

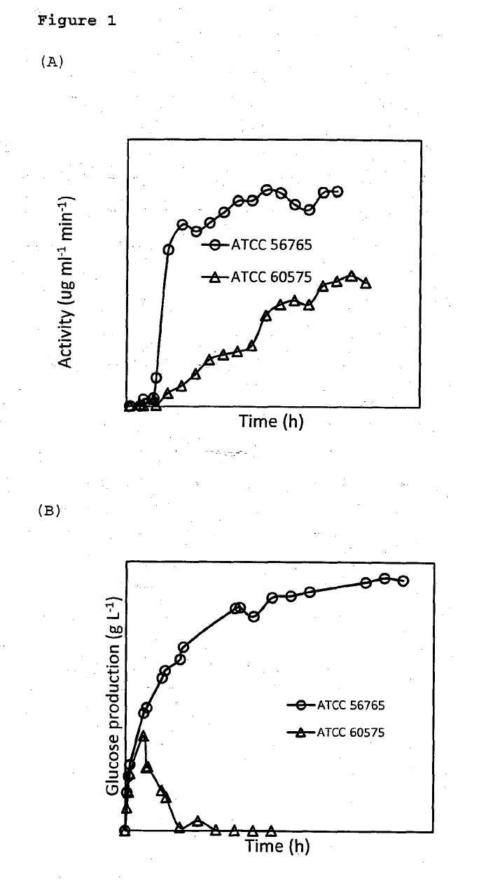

FIG. 1 shows the time courses of cellulase production at (30±1)° C. (A) and cellulose hydrolysis at 50° C. (|B). It is seen from FIG. 1A that for both fungal strains, the cellulase activity rapidly increased with increasing cultivation time and became less changed after reaching a higher level. The strain ATCC 56765 rapidly reached a high cellulase activity within 100 h, while the strain ATCC 60575 achieved a high cellulase activity within 250 h. The strain ATCC 56765 showed a much higher cellulase activity than the strain ATCC 60575.

It is obvious from FIG. 1B that in the case of strain ATCC 56765, glucose was continuously produced with increasing the incubation time at (50±1)° C., indicating that the fungal cells were effectively deactivated at (50±1)° C. In the case of strain ATCC 60575, glucose was rapidly produced within 24 h but became rapidly consumed afterwards with almost no glucose being detected after 72 h. This might be ascribed to the heat tolerance of this fungal strain making it able to digest glucose to grow even at (50±1)° C. Therefore, the strain ATCC 56575 was chosen for the subsequent experiments.

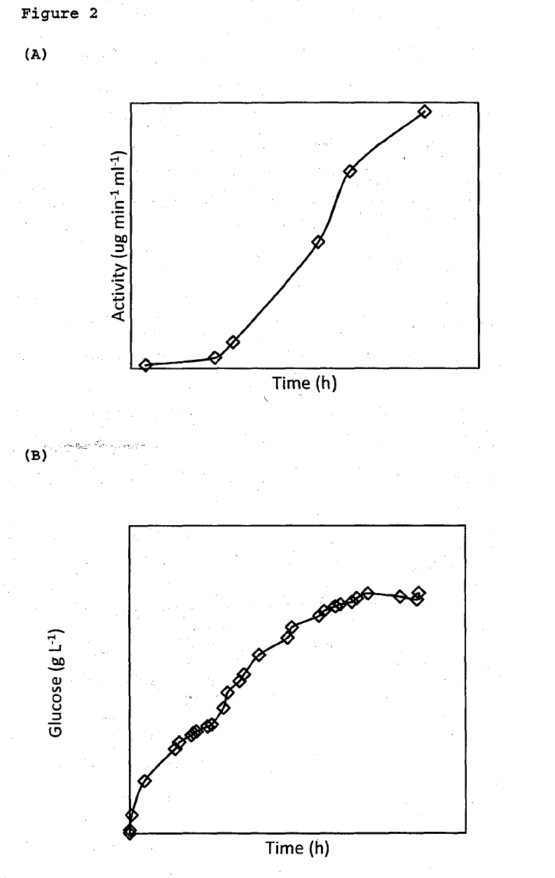

FIG. 2A shows that the cellulase activity rapidly, increased with increasing cultivation time in the fermenter. It was observed that the initial cellulose (20 g/L) was almost completely consumed within 67 h at (30±1)° C., giving the highest cellulase activity. After that, the temperature was raised to (50±1)° C. and 20 g of cellulose was added followed by addition of another 20 g of cellulose at 115 h (FIG. 2B). It is obvious that the glucose concentration rapidly increased with increasing the incubation time and reached 31.2 g/L at 356 h, corresponding to the complete hydrolysis of 30.9 g of cellulose. Afterwards, no further increase in glucose-concentration was observed, although there was still noticeable amount of cellulose remaining In the fermenter, which might be ascribed to the product inhibitions. As the total amount of cellulose used for the cellulase production and cellulose hydrolysis was 60 g, 51% of the total cellulose used was converted to glucose.

### Cellulase Production, Simultaneous Cellulose Hydrolysis and Fermentation to Lactic Acid

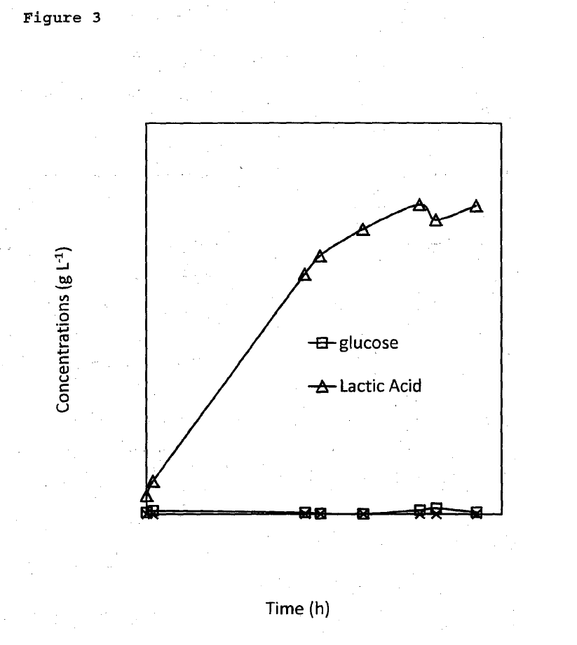

As a high concentration of cellulose was unable to be completely hydrolyzed possibly due to the product inhibitions (FIG. 2B), after the cellulase production in a 2 L fermenter containing 1 L of culture medium for 67 h, the simultaneous cellulose hydrolysis and fermentation to lactic acid using a thermophilic lactic acid bacterium was conducted by raising the temperature to (50±1)° C., adding 80 g of cellulose and some essential nutrients for lactic acid bacteria and inoculating the thermophilic lactic acid bacteria (FIG. 3). The broth pH was kept at 5.5 to compromise the cellulose hydrolysis (optimal pH 5.0) and lactic acid production (optimal pH6.0). As expected, lactic acid was continuously produced with increasing fermentation time. The glucose concentration was maintained at very low levels (<1.5 g/L) during the whole fermentation period, indicating that the glucose inhibition to the cellulose hydrolysis was effectively lessened due to the simultaneous conversion of glucose to lactic acid. The final lactic acid concentration reached 79.2 g/L, corresponding to 71% of conversion of the total cellulose used to lactic acid.

### Discussion

Here the feasibility of the 1-pot, 2-step process for enzymatic hydrolysis of cellulose has been shown using whole cell culture without the requirement of separating the cellulase enzymes making use of the fact that most cellulase-producing fungi survive and produce cellulases only under mesophilic conditions but. the produced cellulases are most active under thermophilic conditions. Trichoderma reesei RUT-C30, one of the best fungal cellulase producers, was chosen as the model strain for the experiments. It is well known that the enzymatic hydrolysis of cellulose to glucose needs the synergistic action of three cellulases, endoglucanase, cellobiohydrolase and p-glucosidase. However, the amount of β-glucosidase secreted by this fungus has been reported to be insufficient for. effective cellulose conversion. Even though, a glucose yield of at least 51% was achieved (FIG. 2B). By the strategy of cellulase production followed by simultaneous cellulose hydrolysis and fermentation to lactic acid, the lactic acid yield reached 71% based on the total cellulose used, corresponding to a glucose yield of 71% as the theoretical yield of lactic acid from glucose is 100%. If a strong β-glucosidase-producer is compatible with Trichoderma reesei RUT-C30 and the two strains are co-cultured to compensate the insufficiency of β-glucosidase secreted by the latter, the glucose yield is expected to be further improved. The use of whole cells for cellulose hydrolysis is expected to significantly reduce the cost of cellulase enzymes. This 1-pot, 2-step process is also applicable for enzymatic hydrolysis of hemicellulose.

### Example 2

Examples of 1-pot, 2-step process for cellulose hydrolysis/fermentation

### Example 2A

The seed Trichoderma reesei RUT-C30 was prepared in 100 ml culture medium at (30±1)° C. for 3 days and 10% (v/v) of inoculum, was added to 1 L culture medium of pH 5.0 in a 2 L fermenter at (30±1)° C. The stirring rate was kept at 300 rpm and the pH was controlled at 3.75 for 48 h and 3.5 afterwards. Filter-sterilized air was continuously bubbled at ca 0.3 L/min to maintain a PO₂ of 20%. After 67 h, the temperature was raised to (50±1)° C. and 20 g of cellulose was added followed by addition of another 20 g of cellulose at 115 h. After 356 h, the glucose concentration reached 31.2 g/L, corresponding to a 51% of cellulose conversion to glucose based on the total cellulose used.

### Example 2B

The seed Trichoderma reesei RUT-C30 was prepared in 100 ml culture medium at (30±1)° C. for 3 days and 10% (v/v) of inoculum was added to 1 L culture medium of pH 5.0 cellulase in a 2 L fermenter at (30±1)° C. The stirring rate was kept at 300 rpm and the pH was controlled at 3.75 for 48 h and 3.5 afterwards. Filter-sterilized air was continuously bubbled at ca 0.3 L/min to maintain a PO₂ of 20%. After 67 h, the temperature was raised to (50±1)° C., 80 g of cellulose and some essential nutrients were added and thermophilic lactic acid bacterium Bacillus coagulans JI-n-12-1-1 was inoculated (10%, v/v). The air supply was stopped and the broth pH was kept at 5.5 during the fermentation. After 115 h, the lactic acid concentration reached 79.2 g/L, corresponding to a 71% of cellulose conversion to lactic acid based on the total cellulose used.

It will be understood that the invention has been described by way of example only and modifications may be made whilst remaining within the scope and spirit of the invention.

1. A method for enzymatic hydrolysis of cellulose using whole cell cultures of cellulase-producing microbes, wherein the method comprises the following steps:
a) cultivating the cellulase-producing microbes at a first temperature to produce cellulases;
b) deactivating the celluase-producing microbes at a second temperature, the second temperature being at a higher temperature level than the first temperature;
c) hydrolysing cellulose to produce glucose using said cellulases; and
d) fermenting the glucose produced in step c) into lactic acid by one or more thermophilic lactic acid bacteria.

2. The method according to claim 1, wherein step c) comprises adding more cellulose into the broth for hydrolysis by said cellulases and/or wherein step c) is performed at the same higher temperature level as step b).

3. The method according to claim 1, wherein the cellulose substrate is provided in the form of one or more of the following: cellulose, hemicellulose, holocellulose and lignocelluloses.

4. The method according to claim 1, wherein the microbes can be cellulase producers, hemicellulase producers or ligninase producers.

5. The method according to claim 1, wherein the cellulase-producing microbes are selected from the group consisting of:
Trichoderma species, Aspergillus species and Penicillium species, Clostridium species and Bacillus species.

6. The method according to claim 1, wherein the microbes are cultivated alone or as a mixture of several strains.

7. The method according to claim 1, wherein the cellulase-producing microbes are Trichoderma reesei RUT-C30 (ATCC 56765) or a variant derived therefrom, optionally in combination with a β-glucosidase producer.

8. The method according to claim 1 wherein the microbes are isolated from nature or obtained from any culture collections.

9. The method according to claim 1, wherein the first temperature is selected from the group consisting of 10-50° C., 15-45° C., 20-40° C., 25-35° C. and 27-33° C.

10. The method according to claim 9 wherein the first temperature is about 30° C.

11. The method according to claim 1, wherein:
(a) the first temperature is selected from the group consisting of: 10-50° C., 15-45° C., 20-40° C., 25-35° C. and 27-33° C.; and
(b) the second temperature is selected from the group consisting of: 45-100° C., 50-100° C., 50-80° C., 50-70° C. or 50-60° C.

12. The method according to claim 9 wherein the first and/or second higher temperature is from 45-55° C.

13. The method according to claim 10 wherein the second higher temperature is about 50° C.

14. The method according to claim 1 wherein the one or more thermophilic lactic acid bacteria comprises or consists of a Bacillus species, preferably B. coagulans.

15. The method according to claim 1 wherein step d) is performed simultaneously with step c).

16. The method according to claim 1 wherein cellulose hydrolysis and glucose fermentation is conducted simultaneously by adding thermophilic lactic acid bacteria and supplementing nutrients to produce lactic acid.

17. The method according to claim 1, wherein one, two or all three of the following conditions are met: (i) steps a) to c) are performed in a single reactor; (ii) steps a) to c) are performed without any separation steps; (iii) steps a) to c) do not comprise the addition of enzymes to the reaction broth.

18. The method according to claim 1, wherein one, two or all three of the following conditions are met: (i) steps a) to d) are performed in a single reactor; (ii) steps a) to d) are performed without any separation steps; (iii) steps a) to d) do not comprise the addition of enzymes to the reaction broth.

19. The method according to claim 1, wherein the method further comprises:
e) recovering one or more products in the form of one or more fermentation products, one or more co-products, or one or more enzymes from the reaction broth.
